(19) United States
(12) Patent Application Publication
(10) Pub. No.: US 2014/0287951 A1
(43) Pub. Date: Sep. 25, 2014

(54) Detection of Auto-Antibodies to Specific Glycans as Diagnostic Tests for Autoimmune Diseases

(71) Applicant: THE JOHNS HOPKINS UNIVERSITY, Baltimore, MD (US)

(72) Inventors: Bruce Scott Bochner, Lutherville, MD (US); Stephan Matthias von Gunten, Bern (CH); Antony Rosen, Pikesville, MD (US); Thomas Grader-Beck, Reisterstown, MD (US)

(73) Assignee: THE JOHNS HOPKINS UNIVERSITY, Baltimore, MD (US)

(21) Appl. No.: 14/215,156

(22) Filed: Mar. 17, 2014

Related U.S. Application Data
(63) Continuation of application No. 13/392,202, filed on Mar. 23, 2012, now abandoned.
(60) Provisional application No. 61/236,657, filed on Aug. 25, 2009.

(51) Int. Cl. G01N 33/564

(52) U.S. Cl. CPC G01N 33/564
USPC 506/9; 435/7.92

(57) ABSTRACT

The invention provides uses, methods, and kits for diagnosing an autoimmune disease, particularly scleroderma and systemic lupus erythematosus, in a subject by detecting the presence of one or more antibodies that specifically bind to one or more glycans in a subject sample.

### CROSS-REFERENCE TO RELATED APPLICATIONS

This application claims priority to U.S. Provisional Patent Application Ser. No. 61/236,657 filed on Aug. 25, 2009. The application is incorporated herein by reference in its entirety.

### BACKGROUND

Systemic Sclerosis (scleroderma or SSc) is an autoimmune disease that is characterized by endothelial cell damage, fibroblast activation, extracellular matrix (ECM) accumulation and abnormal angiogenesis that carries a high rate of morbidity and mortality. The pathogenesis of scleroderma remains unclear, but is thought to involve an autoimmune response against target organs with early production of auto-antibodies and inflammatory mononuclear cell infiltrates followed by loss of organ function and fibrosis. Principal target organs are the skin, the gastrointestinal tract, the lungs and kidneys, although other organs are also frequently involved. A vasculopathy is observed with vasospasm and intimal proliferation of small arteries leading to decreased blood flow in affected organs and hypoxia. Raynaud's phenomenon, a painful discoloration of hands and feet in particular with exposure to cold, is a consequence of such vascular abnormalities. It is observed in about 90-95 percent of scleroderma patients and often results in severely painful non-healing ulcers and digital loss. One of the major causes of mortality is fibrosis of lung tissue (interstitial lung disease) and severe pulmonary hypertension.

Auto-antibodies that recognize membrane-bound and extracellular antigens are thought to play an important role in the pathogenesis of scleroderma, but direct evidence for this hypothesis remains limited. Thus, anti-endothelial cell antibodies (AECA) are found in 22-86 percent of scleroderma patients and have the capacity to modulate endothelial cell function as well as to induce apoptosis. Similarly, anti-fibroblast antibodies (AFA) from sera of patients with scleroderma have been shown to induce proinflammatory and proadhesive properties. The target structures of these antibodies remains largely unknown. Platelet-derived growth factor receptor (PDGF-R) may represent an important auto-antigen in this regard, although the specificity for scleroderma and functional consequence of anti-PDGF-R antibodies remains controversial.

Systemic lupus erythematosus (SLE or lupus) is considered the prototype of systemic autoimmune diseases since its manifestations may involve almost any organ system, ranging from skin to kidneys, the hematopoetic system, joints and the cardiovascular system. The mechanisms underlying the pathogenesis of the disease remain largely unknown. However, the generation of an aberrant immune response represents a central feature in SLE patients and is thought to be a critical factor for the initiation of end-organ damage. In particular, the immune response in SLE produces distinct antibodies (auto-antibodies) that target self-molecules, also called auto-antigens. Auto-antigens are comprised of a heterogeneous and only partly identified group of molecules that are ubiquitously expressed in the body and normally not immunogenic. In SLE, binding of auto-antibodies to exposed auto-antigen in end-organ tissue and/or deposition of antibody-antigen complexes is thought to lead to tissue damage, most likely through complement activation.

### SUMMARY OF THE INVENTION

The invention provides uses of glycans for detection of antibodies to diagnose autoimmune disease; and methods and kits for diagnosing an autoimmune disease in a subject by detecting the presence of one or more antibodies that specifically bind to glycans in a subject sample.

The invention provides for the use of a glycan in the manufacture of an immunoassay for diagnosing an autoimmune disease in a subject including a glycan for contacting with a subject sample; and detecting binding of the antibody in the subject sample to the glycan, wherein binding of the antibody to the glycan is diagnostic for an autoimmune disease. The binding of the antibody from the subject sample to the glycan is specific binding. The immunoassay can include one or more agents or components for the detection of the binding of the antibody from the sera to the glycan. In certain embodiments, the autoimmune disease is scleroderma or systemic lupus erythematosus (SLE). In certain embodiments, when the autoimmune disease is scleroderma and the glycan is one or more (e.g., 1, 2, 3, 4, 5, 6, 7, 8, 9, 10, 11, 12, 13, 14, 14, 16, 17, 18, or 19) of a 4-sulfated LacNAc, a 4-6-disulfated LacNAc, [4OSO3][6OSO3]Galβ1-4GlcNAc; [4OSO3]Galβ1-4GlcNAc; [3OSO3]Galβ1-4[6OSO3]GlcNAc; [6OSO3]Galβ1-4GlcNAc; Galβ1-4GlcNAcβ1-3Galβ1-4Glc; Galβ1-3GlcNAcβ1-3Galβ1-4Glc; Galα1-4Galβ1-4Glc; Galβ1-3GalNAcβ1-3Galα1-4Galβ1-4Glc; Manα1-2Manα1-3(Manα1-2Manα1-6)Man; Manα1-2Manα1-2Manα1-3(Manα1-2Manα1-6(Manα1-3)Manα1-6)Man; Manα1-2Manα1-3Man; Neu5Acα2-3Galβ1-4Glc; Neu5Acα2-8Neu5Acα2-8Neu5Acα2-3(GaINAcβ1-4)Galβ1-4GlcNeu5Acα2-3Galβ1-4GlcNAcβ1-3Galβ1-4GlcNAc; Neu5Acα2-3(GalNAcβ1-4)Galβ1-4GlcNAc; Neu5Acα2-8Neu5Acα2-3(GalNAcβ1-4)Galβ1-4Glc; Neu5Acα2-6Galβ1-4GlcNAc; Neu5Acα2-8Neu5Ac; and Neu5Acα2-3(GalNAcβ1-4)Galβ1-4Glc; or any combination thereof.

In certain embodiments, when the autoimmune disease is scleroderma, the glycan is one or more of 4-sulfated LacNAc, a 4-6-disulfated LacNAc, [4OSO3][6OSO3]Galβ1-4GlcNAc; and [4OSO3]Galβ1-4GlcNAc; or any combination thereof. In certain embodiments, the glycan is diagnostic for one or more conditions such as pulmonary hypertension and sicca; or a combination thereof particularly in a subject with scleroderma. In certain embodiments, the binding of the antibody to the glycan is diagnostic for a less severe Raynaud's phenotype in a subject with scleroderma.

In certain embodiments, when the autoimmune disease is SLE, the glycan is one or more of [4OSO3][6OSO3]Galβ1-4GlcNAc; [4OSO3]Galβ1-4GlcNAc; [3OSO3]Galβ1-4[6OSO3]GlcNAc; [6OSO3]Galβ1-4GlcNAc; Manα1-2Manα1-3Man; Neu5Acα2-3(GalNAcβ1-4)Galβ1-4GlcNAc; Neu5Acα2-8Neu5Acα2-3(GalNAcβ1-4)Galβ1-4Glc; Neu5Acα2-6Galβ1-4GlcNAc; Neu5Acα2-8Neu5Ac; and Neu5Acα2-3(GalNAcβ1-4)Galβ1-4Glc.

In certain embodiments, when the disease is SLE or scleroderma, the glycan is one or more of [4OSO3][6OSO3]Galβ1-4GlcNAc; [4OSO3]Galβ1-4GlcNAc; Manα1-2Manα1-3Man; Neu5Acα2-6Galβ1-4GlcNAc; Neu5Acα2-8Neu5Ac; and Neu5Acα2-3(GalNAcβ1-4)Galβ1-4Glc. In certain embodiments, scleroderma and SLE can be distinguished from each other by the presence of an antibody specific to scleroderma or SLE, that is not present in both scleroderma and SLE.

The sample can be any subject sample that would contain antibodies, for example the sample can be blood, serum, or plasma; or any fraction thereof, e.g., a sample enriched for antibodies or a specific type of immunoglobulin. The binding of the antibodies in the subject sample to a glycan can be compared to the binding of a control sample to the glycan. Control samples include, but are not limited to, normal blood, normal serum, normal plasma, and intravenous immunoglobulin (IVIG); or a combination thereof. Normal blood, serum, and plasma can include blood, serum, or plasma from a single subject, or pooled samples from multiple subjects. Control samples can include a sample enriched for antibodies or a specific type of immunoglobulin

The invention provides methods for diagnosing an autoimmune disease in a subject by contacting a subject sample with a glycan; and detecting binding of an antibody in the subject sample to the glycan, wherein binding of the antibody to the glycan is diagnostic for an autoimmune disease. The binding of the antibody from the subject sample to the glycan is specific binding. In certain embodiments, the method further includes the step of obtaining a subject sample. In certain embodiments, the method further includes providing a diagnosis to a subject.

The methods of the invention include the diagnosis of autoimmune diseases such as scleroderma or systemic lupus erythematosus (SLE).

In certain embodiments, the autoimmune disease is scleroderma and the glycan is one or more of a 4-sulfated LacNAc, a 4-6-disulfated LacNAc, [4OSO3][6OSO3]Galβ1-4GlcNAc; [4OSO3]Galβ1-4GlcNAc; [3OSO3]Galβ1-4[6OSO3]GlcNAc; [6OSO3]Galβ1-4GlcNAc; Galβ1-4GlcNAcβ1-3Galβ1-4Glc; Galβ1-3GlcNAc-3Galβ1-4Glc; Galα1-4Galβ1-4Glc; Galβ1-3GalNAcβ1-3Galα1-4Galβ1-4Glc; Manα1-2Manα1-3(Manα1-2Manα1-6)Man; Manα1-2Manα1-2Manα1-3(Manα1-2Manα1-6(Manα1-3)Manα1-6)Man; Manα1-2Manα1-3Man; Neu5Acα2-3Galβ1-4Glc; Neu5Acα2-8Neu5Acα2-8Neu5Acα2-3(GalNAcβ1-4)Galβ1-4GlcNeu5Acα2-3Galβ1-4GlcNAcβ1-3Galβ1-4GlcNAc; Neu5Acα2-3(GalNAcβ1-4)Galβ1-4GlcNAc; Neu5Acα2-8Neu5Acα2-3(GalNAcβ1-4)Galβ1-4Glc; Neu5Acα2-6Galβ1-4GlcNAc; Neu5Acα2-8Neu5Ac; and Neu5Acα2-3(GalNAcβ1-4)Galβ1-4Glc; or any combination thereof.

In certain embodiments, the autoimmune disease is scleroderma and the glycan is one or more of 4-sulfated LacNAc, a 4-6-disulfated LacNAc, [4OSO3][6OSO3]Galβ1-4GlcNAc; and [4OSO3]Galβ1-4GlcNAc; or any combination thereof.

In certain embodiments, binding of the antibody to the glycan is diagnostic for one or more of pulmonary hypertension and sicca; or a combination thereof, particularly when the subject has been diagnosed with scleroderma. In certain embodiments, binding of the antibody to the glycan is diagnostic for a less severe Raynaud's phenotype in subjects diagnosed with scleroderma.

In certain embodiments, the autoimmune disease is SLE and the glycan is one or more of [4OSO3][6OSO3]Galβ1-4GlcNAc; [4OSO3]Galβ1-4GlcNAc; [3OSO3]Galβ1-4[6OSO3]GlcNAc; [6OSO3]Galβ1-4GlcNAc; Manα1-2Manα1-3Man; Neu5Acα2-3(GalNAcβ1-4)Galβ1-4GlcNAc; Neu5Acα2-8Neu5Acα2-3(GalNAcβ1-4)Galβ1-4Glc; Neu5Acα2-6Galβ1-4GlcNAc; Neu5Acα2-8Neu5Ac; and Neu5Acα2-3(GalNAcβ1-4)Galβ1-4Glc.

In certain embodiments, the autoimmune disease is SLE or scleroderma and the glycan is one or more of [4OSO3][6OSO3]Galβ1-4GlcNAc; [4OSO3]Galβ1-4GlcNAc; Manα1-2Manα1-3Man; Neu5Acα2-6Galβ1-4GlcNAc; Neu5Acα2-8Neu5Ac; and Neu5Acα2-3(GalNAcβ1-4)Galβ1-4Glc. In certain embodiments, scleroderma and SLE can be distinguished from each other by the presence of an antibody specific to scleroderma or SLE, that is not present in both scleroderma and SLE.

The sample can be any subject sample that would contain antibodies, for example the sample can be blood, serum, or plasma; or any fraction thereof, e.g., a sample enriched for antibodies or a specific type of immunoglobulin. The binding of the antibodies in the subject sample to a glycan can be compared to the binding of a control sample to the glycan. Control samples include, but are not limited to, normal blood, normal serum, normal plasma, and intravenous immunoglobulin (IVIG); or a combination thereof. Normal blood, serum, and plasma can include blood, serum, or plasma from a single subject, or pooled samples from multiple subjects. Control samples can include a sample enriched for antibodies or a specific type of immunoglobulin

The invention provides kits for the uses of glycans set forth above or for practicing the methods of the invention. Kits include, for example, one or more glycans such as a 4-sulfated LacNAc, a 4-6-disulfated LacNAc, [4OSO3][6OSO3]Galβ1-4GlcNAc; [4OSO3]Galβ1-4GlcNAc; [3OSO3]Galβ1-4[6OSO3]GlcNAc; [6OSO3]Galβ1-4GlcNAc; Galβ1-4GlcNAcβ1-3Galβ1-4Glc; Galβ1-3GlcNAcβ1-3Galβ1-4Glc; Galα1-4Galβ1-4Glc; Galβ1-3GalNAcβ1-3Galα1-4Galβ1-4Glc; Manα1-2Manα1-3(Manα1-2Manα1-6)Man; Manα1-2Manα1-2Manα1-3(Manα1-2Manα1-6(Manα1-3)Manα1-6)Man; Manα1-2Manα1-3Man; Neu5Acα2-3Galβ1-4Glc; Neu5Acα2-8Neu5Acα2-8Neu5Acα2-3(GalNAcβ1-4)Galβ1-4GlcNeu5Acα2-3Galβ1-4GlcNAcβ1-3Galβ1-4GlcNAc; Neu5Acα2-3(GalNAcβ1-4)Galβ1-4GlcNAc; Neu5Acα2-8Neu5Acα2-3(GalNAcβ1-4)Galβ1-4Glc; Neu5Acα2-6Galβ1-4GlcNAc; Neu5Acα2-8Neu5Ac; Neu5Acα2-3(GalNAcβ1-4)Galβ1-4Glc; Neu5Acα2-3(GalNAcβ1-4)Galβ1-4GlcNAc; and Neu5Acα2-8Neu5Acα2-3(GalNAcβ1-4)Galβ1-4Glc. In certain embodiments, the kit can include one or more control glycans as negative controls. In certain embodiments, the kit can include one or more reagents for performing a binding assay, particularly a binding immunoassay. For example, the kit can include one or more glycans bound to a solid support (e.g., multiwell plate, slide, beads, etc.) In certain embodiments, the kit can include directions for performing the immunoassay and for correlating a result with a particular autoimmune disease.

Other embodiments of the invention are provided by the disclosure infra.

### BRIEF DESCRIPTION OF THE FIGURES

FIG. 1: Recognition of distinct carbohydrates by serum antibodies in scleroderma. (A) Antibody binding ratio (ABR) of pooled serum from 40 patients with scleroderma to 320 glycans determined by glycan array CFG 3.0 as outlined in Materials and Methods. (B) List of glycan structures that are highly recognized by pooled scleroderma serum with an ABR>2 SD above the mean (mean 1.57; SD 1.58). (C) Comparison of ABR between pooled serum from scleroderma and SLE. Scleroderma serum recognizes a distinct set of glycans (ABR>1 SD from mean (dark red); ABR>2 SD from mean (bright red) are indicated).

FIG. 2: Antibodies to 4 sulfated LacNAc are frequent and of high titer in patients with scleroderma. (A) Antibodies to 4-sulfated LacNAc were determined by ELISA as outlined in Materials and Methods in individual sera from 181 patients with scleroderma, 40 disease control patients (SLE) and 40 healthy controls. Arbitrary units (A.U.) values as defined in Materials and Methods are significantly higher in scleroderma patients compared to normal healthy controls (p=0.0058). (B) Correlation between fluorescence values obtained by glycan array and A.U. obtained by ELISA in 17 selected scleroderma patients, r²=0.89

FIG. 3: Preferential recognition of 4- and 4-6 sulfated LacNAc by individual sera from scleroderma patients. (A) ABR to all sulfated glycans present in glycan array CFG 3.1 was determined in 17 preselected sera from patients with scleroderma (9 anti-4 sulfated LacNAc positive/8 anti-4 sulfated LacNAc negative by ELISA). (ABR>mean+1 SD (dark red); and ABR>mean+2SD (bright red) are shown). (B) ABR to LacNAc sulfated at position 3, 4 and/or 6. 4- and 4-6 sulfated LacNAc is highly recognized by scleroderma sera with ABRs up to 119. (C) Confirmation of preferential recognition of 4-sulfated LacNAc compared to 3-sulfated and 6-sulfated LacNAc by ELISA. Results from 26/181 previously identified anti-4 sulfated LacNAc positive scleroderma patients (see FIG. 2A) are shown. (4-sulfated LacNAc; 3-sulfated LacNAc; 6-sulfated LacNAc; 1 of the 27 anti-4 sulfated LacNAc positive sera was not available for further testing)

FIG. 4: Antibodies to 4-sulfated LacNAc are not increased in patients with sicca or pulmonary hypertension independent from a diagnosis of systemic sclerosis. Antibodies to 4-sulfated LacNAc were determined by ELISA as outlined in Materials and Methods in individual sera from 181 patients with scleroderma, 40 patients with primary Sjogren's, 16 patients with SLE and secondary Sjogren's, 12 patients with RA and secondary Sjogren's and 25 patients with primary PH (* p=0.038)

### DEFINITIONS

“Antibody-binding ratio” or “ABR” as used herein for individual glycans is calculated by division of net fluorescence units of samples, typically expressed in arbitrary units, from disease patients by fluorescence units from samples of healthy controls. Such ratios can be determined in quantitative assays that have fluorescence as a read out, e.g., ELISA, glycan assay chip. Similar ratios can be determined using radioactive labels or other quantitatively detectable labels. Such ratios can be determined in comparing single test samples to single control samples, single test samples to pooled control samples, or pooled test samples to pooled control samples.

“Antigenic fragment” and the like are understood as at least that portion of an antigen, e.g., a glycan, capable of inducing an immune response in a subject, or being able to be bound by an antibody present in a subject having or suspected of having an autoimmune disease, particularly scleroderma, Sjogren's syndrome or SLE particularly when the antigen is at least one glycan from the group [4OSO3][6OSO3]Galβ1-4GlcNAc; [4OSO3]Galβ1-4GlcNAc; [3OSO3]Galβ1-4[6OSO3]GlcNAc; [6OSO3]Galβ1-4GlcNAc; Galβ1-4GlcNAcβ1-3Galβ1-4Glc; Galβ1-3GlcNAcβ1-3Galβ1-4Glc; Galα1-4Galβ1-4Glc; Galα1-3GaINAcβ1-3Galα1-4Galβ1-4Glc; Manα1-2Manα1-3(Manα1-2Manα1-6)Man; Manα1-2Manα1-2Manα1-3(Manα1-2Manα1-6(Manα1-3)Manα1-3)Manα1-6)Man; Manα1-2Manα1-3Man; Neu5Acα2-3Galβ1-4Glc; Neu5Acα2-8Neu5Acα2-8Neu5Acα2-3(GalNAcβ1-4)Galβ1-4GlcNeu5Acα2-3Galβ1-4GlcNAcβ1-3Galβ1-4GlcNAc; Neu5Acα2-3(GalNAcβ1-4)Galβ1-4GlcNAc; Neu5Acα2-8Neu5Acα2-3(GalNAcβ1-4)Galβ1-4Glc; Neu5Acα2-6Galβ1-4GlcNAc; Neu5Acα2-8Neu5Ac; and Neu5Acα2-3(GalNAcβ1-4)Galβ1-4Glc. It is understood that the glycan can be a portion of a glycoprotein, a glycolipid, free carbohydrate, etc in vivo as an antigen. It is understood that the glycan may not be able to induce an immune response in a normal (e.g., free from autoimmune disease) subject. However, such an antigen can promote an immune response in an individual that does not recognize the peptide as a self-antigen, or who has dysfunctional immune system such that the antigen is not recognized as self.

As used herein, a “binding assay” is any method that allows for the detection of the presence of an anti-glycan antibody in a sample by binding to the glycan antigen. Exemplary methods include, but are not limited to immunoassays such as enzyme-linked immunosorbent assay (ELISA), radio-immunoassay (RIA), glycan microarrays, immunoprecipitation, such as radioimmunoprecipitation (RIP) assay, dot-blot assay, printed glycan array/chip, and the BIAcore® biosensor assay. Such assays can include the detection of binding directly, or the detection of binding indirectly by use of a competitor or use of the glycan antigen to prevent binding of the antibody to a surface. Binding is frequently detected by the use of a labeled secondary antibody or other reagent including a detectable label. In a preferred embodiment, the binding assay is a quantitative binding assay rather than a qualitative binding assay. Binding assays as used herein can be performed to detect a single immunoglobulin type (e.g., IgG, IgA, IgM, IgE, IgD), or more than one immunoglobulin type.

As used herein, “changed as compared to a control” sample or subject is understood as having a level of the analyte or diagnostic or therapeutic indicator to be detected at a level that is statistically different than a sample from a normal, untreated, or control sample. Control samples include, for example, cells in culture, one or more laboratory test animals, or one or more human subjects, pooled sera or plasma from normal subjects or subjects suffering from a specific disease or condition, commercially available immunoglobulin or serum pools such as the IVIG preparations Sandoglobulin and Privigen (CSL Behring, Bern, Switzerland) and Gamunex (Talecris, Research Triangle Park, N.C.). Sandoglobulin and Privigen (IgG content >98%) contain <2% and <0.01% IgA, respectively, and only traces of IgM, IgD, and IgE (as reported by CSL Behring). Control samples are typically, although need not be, from healthy subjects or subjects not suspected of having a specific disease or condition. Control samples can also be historic samples collected from an earlier time point(s). Control samples can be matched to the subject or subjects being tested for a specific disease or condition using, for example, one or more of the criteria listed in Table 2 (e.g., age, sex, ethnicity, smoking status, disease duration (calculated from the date of onset of first non-Raynaud's phenomenon (RP) symptom), scleroderma subtype, specific organ involvement, and auto-antibody status.). Control samples can be matched, but need not be, matched to test samples, e.g., control samples enriched for IgG are compared to test samples enriched for IgG. Alternatively, the test and control samples can be normalized for the amount of Ig or IgG assayed for binding to the specific glycan. Methods to select and test control samples are within the ability of those in the art. An analyte can be a naturally occurring substance that is characteristically expressed or produced by the cell or organism (e.g., an antibody) or a substance produced by a reporter construct (e.g, β-galactosidase or luciferase). Depending on the method used for detection, the amount and measurement of the change can vary. Changed as compared to a control reference sample can also include a change in one or more signs or symptoms associated with or diagnostic of sclerodema or SLE. Determination of statistical significance is within the ability of those skilled in the art, e.g., the number of standard deviations from the mean that constitute a positive result.

As specific values for control and test samples varies based on a number of factors including, for example, the specific assay method used, the specific test and control samples used, etc. It is understood that binding may be defined in arbitrary units or as a quotient. A sample is considered to be weakly positive for antibody binding to a glycan if the amount of binding is 1-2 SD above the amount of the value of a control, non-disease sample, typically a pooled sample. Binding is defined as at least 2 SD above the amount of the value of a control, non-disease sample, typically a pooled sample. Binding can also be defined as at least 3 SD (or more, e.g., 4 SD, 5 SD, 6 SD, 7 SD, 8 SD, etc.) above the amount of the value of a control, non-disease sample, typically a pooled sample. It is understood that the specificity and sensitivity of an assay can be manipulated by selection of specific cut-off and threshold values and that a single, specific threshold value is not required.

As used herein, “detecting”, “detection” and the like are understood that an assay performed for identification of a specific analyte in a sample, e.g., an antibody in a sample. The amount of analyte or activity detected in the sample can be none or below the level of detection of the assay or method.

By “diagnosing” as used herein refers to a clinical or other assessment of the condition of a subject based on observation, testing, or circumstances for identifying a subject having a disease, disorder, or condition based on the presence of at least one sign or symptom of the disease, disorder, or condition. Typically, diagnosing using the method of the invention includes the observation of the subject for other signs or symptoms of the disease, disorder, or condition in conjunction with the methods provided herein. It is understood that a diagnosis is typically based on more than one criteria (e.g., the presence or absence of more than one clinical observation, the presence or absence of more than one diagnostic marker, or a combination thereof). A test can be diagnostic for a disease or condition without being conclusive as an independent assay. In some instances, diagnosing can include prediction of an exacerbation, flare, or recurrence of symptoms in a disease or condition that has an intermittent pathology, or to detect subclinical disease in a subject.

As used herein, a “glycan” is understood as a polysaccharide or oligosaccharide. Glycan may also be used to refer to the carbohydrate portion of a glycoconjugate, such as a glycoprotein, glycolipid, or a proteoglycan. Glycans usually consist solely of O-glycosidic or N-glycosidic linkages of monosaccharides.

As used herein, an “immunoassay” is any method that detects the specific binding of an antibody to an antigen.

As used herein, “isolated” or “purified” when used in reference to a glycan means that a naturally occurring glycan has been removed from its normal physiological environment (e.g., protein isolated from plasma or tissue) or is synthesized in a non-natural environment (e.g., artificially synthesized in an in vitro translation system with microsomes or using chemical synthesis), and optionally cleaved from a peptide, lipid, or other macromolecule to which it is attached. Thus, an “isolated” or “purified” glycan can be in a cell-free solution or placed in a different cellular environment (e.g., expressed in a heterologous cell type). The term “purified” does not imply that the polypeptide is the only glycan or macromolecule present, but that it is essentially free (about 90-95%, up to 99-100% pure) of cellular or organismal material naturally associated with it, and thus is distinguished from naturally occurring glycan. Similarly, an isolated peptide or nucleic acid is removed from its normal physiological environment. “Isolated” when used in reference to a cell means the cell is in culture (i.e., not in an animal), either cell culture or organ culture, of a primary cell or cell line. Cells can be isolated from a normal animal, a transgenic animal, an animal having spontaneously occurring genetic changes, and/or an animal having a genetic and/or induced disease or condition. An isolated virus or viral vector is a virus that is removed from the cells, typically in culture, in which the virus was produced.

As used herein, “kits” are understood to contain at least one non-standard laboratory reagent for use in the methods of the invention in appropriate packaging, optionally containing instructions for use. The kit can further include any other components required to practice the method of the invention, as dry powders, concentrated solutions, or ready to use solutions. In some embodiments, the kit comprises one or more containers that contain reagents for use in the methods of the invention; such containers can be boxes, ampules, bottles, vials, tubes, bags, pouches, blister-packs, or other suitable container forms known in the art. Such containers can be made of plastic, glass, laminated paper, metal foil, or other materials suitable for holding reagents.

As used herein, a “less severe Raynaud's phenotype” is understood, for example, as a severity score of 1.5±0.7 versus 2.0±0.7 (p=0.016; odds ratio 0.5) in a subject sample. In certain embodiments, a less severe Raynaud's phenotype is understood as a score of 2.0 or less. In certain embodiments, a less severe Raynaud's phenotype is understood as those with scleroderma not having antibodies that specifically bind to a 4-sulfated LacNAc are more likely to have less severe Raynaud's phenotype than those who have antibodies that specifically bind to a 4-sulfated LacNAc.

“Obtaining” is understood herein as manufacturing, purchasing, or otherwise coming into possession of.

As used herein, “operably linked” is understood as joined, preferably by a covalent linkage, e.g., joining an amino-terminus of one peptide, e.g., expressing an enzyme, to a carboxy terminus of another peptide, e.g., expressing a signal sequence to target the protein to a specific cellular compartment; joining a promoter sequence with a protein coding sequence, in a manner that the two or more components that are operably linked either retain their original activity, or gain an activity upon joining such that the activity of the operably linked portions can be assayed and have detectable activity, e.g., enzymatic activity, protein expression activity.

As used herein, “pulmonary arterial hypertension” or “PAH” a subject is considered to have PAH if the estimated right ventricular systolic pressure (eRVSP) determined by Doppler echocardiography is >40 mm Hg in separate tests and there was no overt clinical evidence of congestive heart failure, thromboembolic disease, or severe pulmonary interstitial fibrosis (FVC <50%).

As used herein, “plurality” is understood to mean more than one. For example, a plurality refers to at least two, three, four, five, or more.

A “sample” as used herein refers to a biological material that is isolated from its environment (e.g., blood or tissue from an animal, cells or material from cells, or conditioned media from tissue culture) and is suspected of containing, or known to contain an analyte, such as an auto-antibody. A sample can also be a partially purified fraction of a tissue or bodily fluid. Bodily fluids include, but are not limited to blood and fractions thereof, e.g., serum, plasma, red blood cells, white blood cells and subsets thereof, urine, saliva, tears, lymph, and spinal fluid. A reference sample can be a “normal” sample, from a donor not having the disease or condition fluid, or from a normal tissue in a subject having the disease or condition. A reference sample can also be from an untreated donor or cell culture not treated with an active agent (e.g., no treatment or administration of vehicle only). A reference sample can be a pooled sample from a plurality of normal or not normal subjects. A reference sample can also be taken at a “zero time point” prior to contacting the cell or subject with the agent or therapeutic intervention to be tested.

“Sensitivity and specificity” are statistical measures of the performance of a binary classification test. The sensitivity (also called recall rate in some fields) measures the proportion of actual positives which are correctly identified as such (e.g. the percentage of sick people who are identified as having the condition); and the specificity measures the proportion of negatives which are correctly identified (e.g. the percentage of well people who are identified as not having the condition). They are closely related to the concepts of type I and type II errors. A theoretical, optimal prediction can achieve 100% sensitivity (i.e. predict all people from the sick group as sick) and 100% specificity (i.e. not predict anyone from the healthy group is sick).

The concepts are expressed mathematically as follows:

sensitivity=#true positives/(#true positives+#false negatives)

specificity=#true negatives/(#true negatives+#false positives).

A diagnostic test with a high sensitivity and a low specificity, or a low sensitivity and high specificity can be useful diagnostically in combination with one or more other tests, preferably that have different properties in relation to sensitivity and specificity.

An agent, antibody, polypeptide, nucleic acid, or other compound “specifically binds” a target molecule, e.g., antigen, polypeptide, nucleic acid, or other compound, when the target molecule is bound with at least 100-fold, preferably at least 500-fold, preferably at least 1000-fold, preferably at least a 5000-fold, preferably at least a 10,000-fold preference as compared to a non-specific compounds, or a pool of non-specific compounds. Specifically binds can be used in relation to binding one of two or more related compounds that have physically related structures. Binding preferences and affinities, absolute or relative, can be determined, for example by determining the affinity for each pair separately or by the use of competition assays or other methods well known to those of skill in the art.

“Sicca” is a condition of persistent dry eyes and/or dry mouth for at least three months. Sicca affecting the eye, includes dryness of the eyes with a sensation of sandy/gritty eyes, or requiring eye drops 3 times or more per day. Sicca affecting the mouth includes dryness, swollen salivary glands, or requiring liquids to swallow

A “subject” as used herein refers to living organisms. In certain embodiments, the living organism is an animal. In certain preferred embodiments, the subject is a mammal. In certain embodiments, the subject is a domesticated mammal or a primate including a non-human primate. Examples of subjects include humans, non-human primates, monkeys, dogs, cats, mice, rats, cows, horses, goats, and sheep. A human subject may also be referred to as a patient.

A subject “suffering from or suspected of suffering from” a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition, or syndrome. Methods for identification of subjects suffering from or suspected of suffering from autoimmune diseases such as scleroderma or SLE is within the ability of those in the art. Subjects suffering from, and suspected of suffering from, a specific disease, condition, or syndrome are not necessarily two distinct groups.

As used herein, “susceptible to” or “prone to” or “predisposed to” a specific disease or condition and the like refers to an individual who based on genetic, environmental, health, and/or other risk factors is more likely to develop a disease or condition than the general population. An increase in likelihood of developing a disease may be an increase of about 10%, 20%, 50%, 100%, 150%, 200%, or more.

As used herein, “under conditions to allow the antibody to bind to the glycan” is understood as conditions under which immunoassays such as ELISA, immunoblot, glycan array, immunoprecipitation, BIACORE, or other immunoassays are performed. Such conditions are well known in the art and provided, for example in Antibodies: A Laboratory Manual (Eds. E. Harlow and D. Lane, Cold Spring Harbor Laboratory, copyright 1988) and in versions of Current Protocols in Molecular Biology (Eds. F. M. Ausubel et al., copyright Wiley) available prior to the priority date of the instant application. For example conditions that allow the antibody to bind the glycan include a pH of about 6 to about 8, a salt concentration, typically a sodium concentration, of about 100-300 mM, typically in the presence of one or more buffers, a detergent, particularly a non-ionic detergent, and a blocking agent to prevent non-specific binding of proteins to the glycan. It is understood that antibodies bind specifically to their antigens at pH and salt concentrations outside of those specifically provided. It is further understood that binding of an antibody to an antigen can be determined empirically and optimized for binding to a specific antigen or antibody. It is understood that the conditions to allow binding may vary depending on the type of immunoassay or device used to detect binding.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

More than one is understood as 2, 3, 4, 5, 6, 7, 8, 9, 10, 11, 12, 13, 14, 15, 16, 17, 18, 19, 20, 25, 30, 40, 50, 100, etc., or any value therebetween.

Unless specifically stated or obvious from context, as used herein, the term “or” is understood to be inclusive.

Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### DETAILED DESCRIPTION OF THE INVENTION

Autoimmune diseases are a spectrum of serious disorders that can be difficult to diagnose and treat. The evaluation of such patients usually requires a panel of blood tests to help distinguish between related conditions and confirm other clinical and diagnostic findings. The availability of novel blood tests to aid in this process would improve the ability to reliably diagnose such conditions.

Differential glycosylation of proteins and lipids plays an important role in the regulation of numerous physiological processes. The endothelium and extracellular matrix, two key tissue components affected in scleroderma, contain various highly glycosylated structures (glycans). A number of intracellular and extracellular target auto-antigens are seen with SLE. As demonstrated herein, the immune response in scleroderma and SLE target distinct glycans, leading to the development of specific anti-glycan antibodies.

Batches of serum pooled from patients with the autoimmune disorder SLE or scleroderma were used to screen for the presence of antibodies recognizing a panel of over 300 different glycans using a printed glycan array. Results were compared to pooled sera from 40 healthy control subjects. Binding was quantified by fluorescence using an anti-human IgG detection-reagent. It was discovered that, compared to normal pooled serum, SLE and scleroderma patients had antibodies that recognized the following glycans that have distinct structures:

As shown in Table 1 below, some glycans are diagnostic for scleroderma, some glycans are diagnostic for SLE (i.e., can be used to differentiate scleroderma from SLE), and some glycans are diagnostic for both. Due to the varied presentations of both scleroderma and SLE, it is expected that antibodies to the glycans in Table 1 will be high specificity markers for disease, likely with a relatively low sensitivity as is common with markers for both scleroderma and SLE.

| | |
|---|---|
| SSc | SLE |

Sulfated glycans

| | | |
|---|---|---|
| [4OSO3][6OSO3]Galβ1-4GlcNAc | + | + |
| [4OSO3]Galβ1-4GlcNAc | + | + |
| [3OSO3]Galβ1-4[6OSO3]GlcNAc | | + |
| [6OSO3]Galβ1-4GlcNAc | | + |

Galactosylated glycans

| | |
|---|---|
| Galβ1-4GlcNAcβ1-3Galβ1-4Glc | + |
| Galβ1-3GlcNAcβ1-3Galβ1-4Glc | + |
| Galα1-4Galβ1-4Glc | + |
| Galβ1-3GalNAcβ1-3Galα1-4Galβ1-4Glc | + |

Mannosylated glycans

| | | |
|---|---|---|
| Manα1-2Manα1-3(Manα1-2Manα1-6)Man | + | |
| Manα1-2Manα1-2Manα1-3(Manα1-2Manα1-6(Manα1-3) | + | |
| Manα1-6)Man | | |
| Manα1-2Manα1-3Man | + | + |

Sialylated glycans

| | | |
|---|---|---|
| Neu5Acα2-3Galβ1-4Glc | + | |
| Neu5Acα2-8Neu5Acα2-8Neu5Acα2-3(GalNAcβ1- | | + |
| 4)Galβ1-4GlcNeu5Acα2-3Galβ1-4GlcNAcβ1-3Galβ1- | | |
| 4GlcNAc | | |
| Neu5Acα2-3(GalNAcβ1-4)Galβ1-4GlcNAc | | + |
| Neu5Acα2-8Neu5Acα2-3(GalNAcβ1-4)Galβ1-4Glc | | + |
| Neu5Acα2-6Galβ1-4GlcNAc | (+) | + |
| Neu5Acα2-8Neu5Ac | (+) | + |
| Neu5Acα2-3(GalNAcβ1-4)Galβ1-4Glc | (+) | + |

Based on the identification of antibodies to a number of glycans, a subsequent study was performed using an ELISA assay on individual subject samples for antibodies to sulfated LacNAc, a dominant antibody identified in scleroderma patient using the array. Antibodies to sulfated LacNAc were determined in 181 individual sera from scleroderma patients and 40 healthy controls by ELISA using an anti-human Fc gamma specific secondary antibody for detection, a high-titer positive scleroderma serum as standard and a cut-off of 3SD above healthy controls. Statistical analysis of clinical associations was performed using student's t-test and logistic regression.

Immunogenicity was predominantly conferred by sulfation at position 4 of galactose (4S-LacNAc), but not at position 3 or 6 (13.6 fold; 3.0 fold and 0.9 fold compared to controls; respectively). 27/181 (14.9%) patients were positive for anti-4S-LacNAc antibodies by ELISA compared to 1/40 (2.5%) of healthy controls. Anti-4S-LacNAc positive scleroderma patients had a higher prevalence of pulmonary hypertension as determined by a right ventricular systolic pressure (RVSP)>40 mm Hg on echocardiogram (15/27; 55.7% versus control 49/154; 31.8% p=0.02). The odds ratio for pulmonary hypertension was 2.6 (1.1, 6.3) after adjusting for age, gender, race and disease type. Anti-4S-LacNAc positive patients accounted for 23.4% of all patients with pulmonary hypertension. There was no association between anti-48-LacNAc positivity and disease type (limited versus diffuse scleroderma) or the presence of anti-topoisomerase I or anti-centromere antibodies.

This study demonstrates that sera from scleroderma patients contain high titer IgG antibodies targeting glycan structures, with sulfated N-Acetyl-lactosamine (LacNAc) as a dominant target of the antibody response in scleroderma. The antibodies preferentially target LacNAc sulfated at the 4 position.

Further the 4S-LacNAc was identified as a frequent target of the antibody response in scleroderma and antibody positivity was associated with echocardiographic evidence of pulmonary hypertension. The invention provides a new antigen for use in the diagnosis of scleroderma, particularly scleroderma with pulmonary hypertension.

Without being bound by mechanism, these results suggest that specific posttranslational carbohydrate modifications may be an important immunogen in scleroderma. Such antibodies would be prime candidates to interfere with glycosylation-dependent processes and thus may play an important role in the pathogenesis of the disease.

Further, the data provided herein demonstrate that detection of antibodies against glycans can be used as a diagnostic and/or prognostic test for identifying, monitoring, or categorizing patients with the autoimmune diseases such as lupus, scleroderma or other autoimmune diseases. Testing for these serum antibodies can be performed by any method, typically a binding assay, known to those skilled in the art including, but not limited to, enzyme-linked immunosorbent assay (ELISA), radio-immunoassay (RIA), glycan microarrays, immunoprecipitation, such as radioimmunoprecipitation (RIP) assay, and the BIAcore® biosensor assay. The specific method of detection of one or more antibodies targeted to a glycan as an indicator of an autoimmune disease is not a limitation of the invention.

### Scleroderma

The diagnosis of scleroderma is primarily based on clinical findings, including the presence of characteristic skin changes (sclerodactyly), digital pitting scars of the finger tips or loss of substance of the distal finger pad and the presence of lung fibrosis. At present, there are no reliable serological biomarkers available that would enable clinicians to make the diagnosis of scleroderma before significant organ damage has occurred. Furthermore, scleroderma is characterized by periods of disease flares and remission. Determination of early disease activity is difficult at present and substantial organ damage may occur before a flare can be diagnosed. Some patients may suffer from ongoing smoldering disease activity that escapes clinical detection, but leads to serious consequences. Thus, patients may experience an ongoing inflammatory lung process that slowly progresses to end stage lung disease and premature death. Currently, there are no reliable serological biomarkers available that would help clinicians to determine disease activity in scleroderma.

Auto-antibodies are thought to play an important part in the disease pathogenesis. However, as the presentation of scleroderma varies widely, it is likely that no single auto-antibody or other marker will be present in all subjects with the disease. The identification of biomarkers with regards to auto-antibodies has been focusing on antinuclear antibodies (ANAs) which are a broad class of antibodies present in a number of autoimmune diseases in addition to scleroderma (e.g., SLE). ANAs are found in almost all scleroderma patients, and antibodies against topoisomerase I (ScI70) are highly specific for the disease (approaching 100 percent). However, anti-topoisomerase I antibodies are present in about 40 percent of patients. Similarly, anti-centromere antibodies are associated with the limited scleroderma disease phenotype, but are also found only in a small percentage of patients (about 20-40 percent). Therefore, it is well recognized in the art that detection of an auto-antibody specific for a particular disease can be useful for diagnosis of the disease, even if the auto-antibody is present in only a small portion of subjects with the disease. That is, highly specific markers can be useful for diagnosis even when they are not highly sensitive. As demonstrated herein, antiglycan auto-antibodies are useful for diagnosis and determination of disease activity.

Efforts to identify auto-antigens in scleroderma and other systemic autoimmune diseases have long been focusing on protein and/or nucleic acid antigens. To our knowledge, this is the first report that provides a comprehensive evaluation of antibody binding to diverse carbohydrate antigens in patients with scleroderma and SLE. Here, we identify a number of glycans useful for the diagnosis of scleroderma and SLE, particularly 4-sulfated LacNAc as a prominent target of the immune response in scleroderma and SLE. Further provided herein is the clinical association of the antibody in scleroderma patients with echocardiographic evidence of pulmonary vascular disease and sicca.

The anti-4 sulfated LacNAc antibodies identified herein (e.g., antibodies that bind [4OSO3][6OSO3]Galβ1-4GlcNAc or [4OSO3]Galβ1-4GlcNAc) in scleroderma patients showed remarkable specificity to the position of the sulfate within the LacNAc structure. The antibodies are of the IgG isotype, as demonstrated by using an anti-human Fc-gamma specific secondary antibody for detection, as well as retention of anti 4-sulfated LacNAc reactivity in the isolated IgG fraction from patient serum (data not shown).

Furthermore, anti-[6OSO3]Galβ1-4GlcNAc; anti-Galβ1-4GlcNAcβ1-3Galβ1-4Glc; anti-Galβ1-3GlcNAcβ1-3Galβ1-4Glc; anti-Galα1-4Galβ1-4Glc; anti-Galβ1-3GalNAcβ1-3Galα1-4Galβ1-4Glc; anti-Manα1-2Manα1-3(Manα1-2Manα1-6)Man; anti-Manα1-2Manα1-2Manα1-3(Manα1-2Manα1-6(Manα1-3)Manα1-6)Man; and anti-Manα1-2Manα1-3Man antibodies were identified in scleroderma patient serum. Additional antibodies that weakly bound Neu5Acα2-6Galβ1-4GlcNAc; Neu5Acα2-8Neu5Ac; and Neu5Acα2-3(GalNAcβ1-4)Galβ1-4Glc were also identified in serum from scleroderma patients. Anti-Manα1-2Manα1-3Man antibodies were also found in patients with SLE. The carbohydrate antigens can be further characterized for binding to patient sera using routine methods such as those provided herein (e.g., testing individual sera for binding to glycans, testing of sera for binding to related glycans, etc.). Such analysis can be used to determine the specificity and sensitivity or each of the antigens demonstrated herein to be useful as a diagnostic marker for an autoimmune disease, particularly scleroderma. Such methods are well known in the art.

The invention provides methods for use of the glycans listed above to detect antibodies from subject samples for the diagnosis if autoimmune disease such as scleroderma, and optionally to stratify subjects diagnosed with scleroderma (e.g., pulmonary involvement, sicca, Raynaud's phenotype). It is understood that the invention includes method and uses for detecting antibody binding to more than one of the glycans demonstrated herein to be associated with scleroderma. It is understood that the methods and uses provided herein can be combined with one or more diagnostic indicators, e.g., immunoassays for previously identified auto-antigens (e.g., nuclear antigens), physical exam, etc., for diagnosing scleroderma such as those provided in the guidelines and diagnostic methods for the diseases and those provided herein.

The pathway by which the anti-glycan antibodies develop, or the physiological effect that these antibodies might have on the subject suffering from SLE or scleroderma, does not limit their use as diagnostic markers. Antibodies to carbohydrate structures were previously thought to develop in a T-lymphocyte independent manner, however, this paradigm has recently been shifting. It has long been known that anti-ABO blood group antibodies, another form of anti-carbohydrate antibodies, are responsible for transfusion reactions in mismatched blood. Whether anti-4 sulfated LacNAc antibodies target self structures and thus represent true auto-antibodies needs to be studied further. However, the pathological effect, or lack thereof; of the anti-glycan antibodies has no effect on their use as diagnostic markers of disease.

Sulfation is a common modification of endogenous human carbohydrate structures. Examples include sulfation of Gal at position 4 in the carbohydrate-protein linkage region of glycosaminoglycans (GAGs), with the sequence GlcAβ1-3Galβ1-3Galβ1-4Xylβ. However, the sulfated Gal in this instance is β1-3 linked to a second Gal rather than β1-4 linked to GlcNAc. 4-sulfation of N-acetylated Gal (GalNAc) represents an important modification in several GAGs, including chondroitin sulfate A and Dermatan sulfate. GAGs are prominently expressed in the extracellular matrix and on endothelial cells, where they regulate processes that may be involved in the pathogenesis of systemic sclerosis, including wound healing, angiogenesis and lymphocyte migration.

Several auto-antibodies have been found to be associated with pulmonary hypertension in scleroderma, including anti-centromere and anti-U3 RNP, with a frequency of about 22 and 17 percent of patients affected, respectively. Anti-endothelial cell antibodies have also been reported in association with pulmonary hypertension, but the targets of these antibodies remain ill defined. In our study, over one half of anti-4 sulfated LacNAc positive patients (55.6%) had echocardiographic evidence of pulmonary vascular disease. Furthermore, anti-4 sulfated LacNAc antibody positivity identified almost a quarter (23.4%) of all scleroderma patients with pulmonary vascular disease. This provides a valuable biomarker to screen for pulmonary hypertension in scleroderma. Interestingly, an increased anti-4 sulfated LacNAc antibody positivity was not found in patients with primary pulmonary hypertension (PPH). This finding is in agreement with the notion that IgG antibodies from patients with idiopathic or scleroderma associated PAH may express distinct reactivity profiles with regards to endothelial cell antigens and that autoimmune and inflammatory pathways likely play a more dominant role in scleroderma-related PAH versus PPH.

Interestingly, anti-4 sulfated LacNAc positivity was associated with pulmonary vascular disease, but a less severe Raynaud's phenotype. Since both disease manifestations are represented on a histopathological level by vasculopathy, one may expect a concordance in severity. However, while an association between severity of Raynaud's syndrome and development of pulmonary hypertension has been found by some investigators, that has not been seen by others.

### Systemic Lupus Erythematosis

At present, the diagnostic process for SLE is cumbersome and involves a set of 11 clinical and laboratory parameters, at least 4 of which should be present in order to make the diagnosis (current guidelines by the American College of Rheumatology, ACR). The relevance of determining auto-antibodies for the diagnosis of SLE is illustrated by the fact that the presence of distinct serum auto-antibodies (anti-double stranded DNA, anti-Sm and anti-Cardiolipin) is so specific for SLE that these tests are utilized in the current ACR classification. However, auto-antibodies to these antigens are present only in a fraction of SLE patients. Thus, at present there is no single laboratory test available that can determine whether a patient has SLE or not.

The immune response in SLE produces distinct auto-antibodies. The target auto-antigens are a heterogeneous and only partly identified group of molecules that are ubiquitously expressed in the body and normally not immunogenic. In SLE, binding of auto-antibodies to exposed auto-antigen in end-organ tissue and/or deposition of antibody-antigen complexes is thought to lead to tissue damage, most likely through complement activation. However, the mechanism of action of the auto-antibodies in disease pathology are not limiting to the instantly invention.

Disease activity in SLE patients is typically characterized by flares and remission. The prediction of flares and the early diagnosis of a flare are of utmost importance to prevent irreversible end-organ damage and decrease mortality. Studies to date have failed to show a reliable correlation between measures of disease activity and the presence or titers of known auto-antibodies. At present, there is no reliable biomarker available to assess early disease activity in SLE.

As part of a pilot project to expand our search for new biomarkers in SLE, pooled sera from SLE patients was screened by the Consortium for Functional Glycomics, a National Institute of General Medical Sciences (NIGMS)-funded project. The screen involved testing for the presence of antibodies capable of binding to a panel of approximately 200 different glycans, When this analysis was performed and compared to binding patterns of a commercially available pool of antibodies (intravenous gamma globulin, or IVIG) used therapeutically, four specific sulfated glycans emerged as being detected by antibodies in lupus sera but not in normal pooled human WIG. These data indicated that SLE patients uniquely make antibodies to these glycans and that detection of these antibodies can be used as a diagnostic and/or prognostic test for SLE.

Based on the initial results, pooled sera from SLE and scleroderma patients was screened by the Consortium for Functional Glycomics using a later version of the glycan library with additional members. The screen involved testing for the presence of antibodies capable of binding to a panel of over 300 different glycans. When this analysis was performed and binding patterns were compared to IVIG as well as a pool of normal serum samples, numerous specific sulfated, galactosylated, mannosylated and sialylated glycans were detected by antibodies in either SLE or scleroderma sera, but not in normal sera (see Table 1). Specifically, SLE samples had antibodies that specifically bound to [4OSO3][6OSO3]Galβ1-4GlcNAc; [4OSO3]Galβ1-4GlcNAc; [3OSO3]Galβ1-4[6OSO3]GlcNAc; [6OSO3]Galβ1-4GlcNAc; Manα1-2Manα1-3Man; Neu5Acα2-3(GalNAcβ1-4)Galβ1-4GlcNAc; Neu5Acα2-8Neu5Acα2-3(GalNAcβ1-4)Galβ1-4Glc; Neu5Acα2-6Galβ1-4GlcNAc; Neu5Acα2-8Neu5Ac; and Neu5Acα2-3(GalNAcβ1-4)Galβ1-4Glc. The carbohydrate antigens can be further characterized for binding to patient sera using routine methods such as those provided herein (e.g., testing individual sera for binding to glycans, testing of sera for binding to related glycans, etc.). Such analysis can be used to determine the specificity and sensitivity or each of the antigens demonstrated herein to be useful as a diagnostic marker for an autoimmune disease, particularly SLE. Such methods are well known in the art.

The invention provides methods for use of the glycans listed above to detect antibodies from subject samples for the diagnosis if autoimmune disease such as SLE, and optionally to stratify subjects diagnosed with SLE. It is understood that the invention includes method and uses for detecting antibody binding to more than one of the glycans demonstrated herein to be associated with SLE. It is understood that the methods and uses provided herein can be combined with one or more diagnostic indicators, e.g., immunoassays for previously identified auto-antigens (e.g., nuclear antigens, cardiolipin, Sm antigens) physical exam, etc., for diagnosing SLE such as those provided in the guidelines and diagnostic methods for the diseases and those provided herein.

### Example 1

### Material and Methods

### Patients

One hundred eighty-one scleroderma patients were randomly identified in the Johns Hopkins scleroderma Center database. All patients met the American College of Rheumatology criteria for scleroderma and were classified as having diffuse cutaneous scleroderma or limited cutaneous scleroderma depending on the extent of skin involvement. Serum from 40 consecutive patients with SLE as well as 20 SLE patients with sicca symptoms, 25 patients with primary pulmonary hypertension diagnosed according to the criteria of the National Institutes of Health Patient Registry for the Catheterization of Primary Pulmonary Hypertension (Rich S D et al. Primary pulmonary hypertension: a national prospective study. Ann Intern Med. 1987; 107:216-223) and 40 healthy controls were also obtained for this study. SLE patients met the 1997 revised ACR criteria for SLE, and primary Sjogren's patients met the San Diego criteria for Sjogren's disease (Fox-RI at al., Sjögren's syndrome. Proposed criteria for classification. Arthritis Rheum. 1986; 29:577-85). Written informed consent was obtained from all patients prior to this study at the time of sample collection. The present study was approved by the Johns Hopkins Institutional Review Board.

### Clinical Phenotyping

Demographic and clinical data, including age, sex, ethnicity, smoking status, disease duration (calculated from the date of onset of first non-Raynaud's phenomenon (RP) symptom), scleroderma subtype, specific organ involvement, and auto-antibody status, were previously recorded for each patient at the time of the clinical visit. RP activity and presence of digital ischemia were determined using a previously published severity score (Medsger, et al., A disease severity scale for systemic sclerosis: development and testing. J Rheumatol, 1999. 26(10): p. 2159-67) (0=no RP, 1=RP with or without vasodilator required, 2=digital pitting scars, 3=digital tip ulcerations, 4=digital gangrene). Active digital ischemia was defined as an RP severity score of 3. Skin involvement was scored according to the modified Rodnan skin thickness score (MRSS [range 0-51]) (Clements et al., Inter and intraobserver variability of total skin thickness score (modified Rodnan TSS) in systemic sclerosis. J Rheumatol, 1995. 22(7): p. 1281-5. Internal organ involvement was assessed using previously published criteria (Medsger, et al, 1999). Pulmonary involvement was determined based on abnormal findings on pulmonary function tests (PFTs) forced vital capacity [FVC] and single-breath diffusing capacity for carbon monoxide [DLCO], measured as the absolute value as well as the percent predicted value for race, sex, and age, according to the American Thoracic Society recommendations (Lung function testing: selection of reference values and interpretative strategies. American Thoracic Society. Am Rev Respir Dis, 1991. 144(5): p. 1202-18). For the purpose of this study, a patient was considered to have evidence of PAH if the estimated right ventricular systolic pressure (eRVSP) determined by Doppler echocardiography was >40 mm Hg in separate tests and there was no overt clinical evidence of congestive heart failure, thromboembolic disease, or severe pulmonary interstitial fibrosis (FVC <50%). This assumption has been supported and confirmed in other studies (Denton, et al., Comparison of Doppler echocardiography and right heart catheterization to assess pulmonary hypertension in systemic sclerosis. Br J Rheumatol, 1997. 36(2): p. 239-43). Lung severity score 0=Normal 1=FVS/DLCO 70-80% predicted or rales or CXR fibrosis 2=50-69% predicted or Mild PHT (RVSP 36-45) 3=<50% predicted or mild-severe PHT (RVSP >45). Heart, gastrointestinal, renal, or musculoskeletal involvement was considered present when the relative Medsger severity score (Medsger, et al, 1999) was 1. Evidence of sicca complex was determined by clinical criteria as daily dry eyes and/or dry mouth for at least 3 months.

### Glycan ELISA

Ninety-six well microtiter Neutravidin plates preblocked with Superblock® (Pierce®) were coated with biotinylated glycans (Lectinity®) as indicated at 200 ng/well in PBS for 2 hours at room temperature. After extensive washes, sera from disease patients and healthy controls were added at 1:250 dilution in PBS 1% BSA and incubated for 1 hour, followed by extensive washes and incubation with a secondary anti-human Fc gamma-specific antibody (JacksonImmuno) diluted 1:10,000 in PBS 1% BSA for 30 minutes. Plates were again washed extensively followed by incubation with SureBlue® (KPL®). The reaction was stopped after 4 minutes and the absorbance was read at 450 nm and 690 nm for reference. O.D. values were transformed into Arbitrary units (A.U.), based on a standard curve established using O.Ds. from serial dilutions of a high-titer positive scleroderma serum (FW-555) after logarithmic transformation. Sera that resulted in an O.D. above the highest standard curve value were diluted further until the O.D. was in the linear range of the assay. ELISAs using other glycans, including the glycans provided herein as well as other glycans, can be performed in a similar manner.

### Glycan Microarray

The glycan microarrays from the CFG (www.functionalglycomics.org/static/consortium/resources/resourcecoreh11.shtml) are prepared from amine functionalized glycan structures covalently coupled in microarrays to N-hydroxysuccinimide-derivatized microscope slides as previously described (Blixt, at al., Printed covalent glycan array for ligand profiling of diverse glycan binding proteins. Proc Natl Acad Sci USA, 2004. 101(49): p. 17033-8.). Pooled and individual sera from patients with disease and healthy controls were analyzed for binding to version 3.0 or 3.1 of the printed array (320 and 377 glycans, respectively) measured by net fluorescence units. The antibody-binding ratio (ABR) for individual glycans was calculated by division of net fluorescence units of samples from disease patients by fluorescence units from samples of healthy controls.

### Statistical Analysis

Statistical significance for the results of the ELISA studies was calculated by Mann-Whitney test. Association of disease, sociodemographic characteristics and odds ratios were calculated by student's test and logistic regression.

### Example 2

### Presence of Distinct Anti-Carbohydrate Antibodies in SLE

Based on the concept that anti-carbohydrate antibodies may serve as biomarkers for human diseases, experiments were performed to identify antibodies in the serum of patients with lupus that uniquely recognize a distinct pattern of naturally occurring glycans. Using a pool of sera generated from 40 SLE patients, an extensive panel (approximately 200) of carbohydrate-based structures (“glycans”) was screened for antibody binding through participation in the Consortium for Functional Glycomics. The library of biotinylated, synthetic and natural ligands is attached by a spacer onto a steptavidin-coated surface. Each unique glycan occupies a set of defined addresses on the array in replicate wells. information on the structures included in the array can be found on the world wide web at www.functionalglycomics.org/static/consortium/resources/resourcecoreh11.shtml. The binding of pooled sera from lupus patients to the glycan array was performed using their standard high throughput screening protocol. In this screen, lupus sera, unlike IVIG, distinctly bound the following glycan structures: [4OSO3][6OSO3]Galβ1-4GlcNAc; [4OSO3]Galβ1-4GlcNAc; [3OSO3]Galβ1-4[6OSO3]GlcNAc; and [6OSO3]Galβ1-4GlcNAc.

These data demonstrate that lupus patients, but not normal donors, make antibodies to non-sialylated, sulfated galactosylated glycans residues. Therefore, detection of these anti-glycan antibodies can be used in identifying, monitoring, or categorizing patients with autoimmune diseases such as lupus.

### Example 3

### Presence of Distinct Anti-Carbohydrate Antibodies in Scleroderma

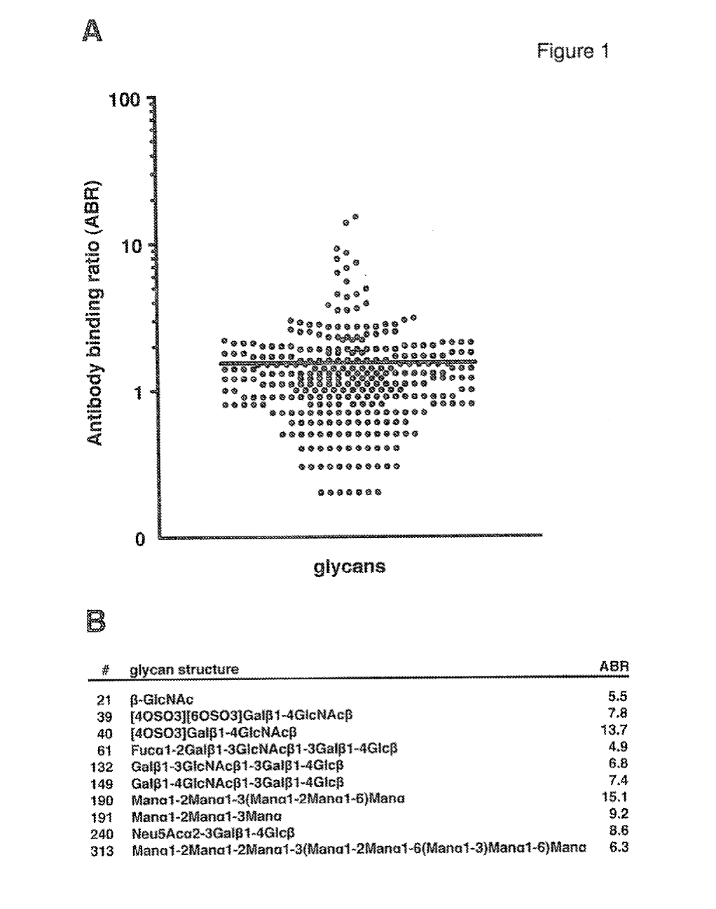

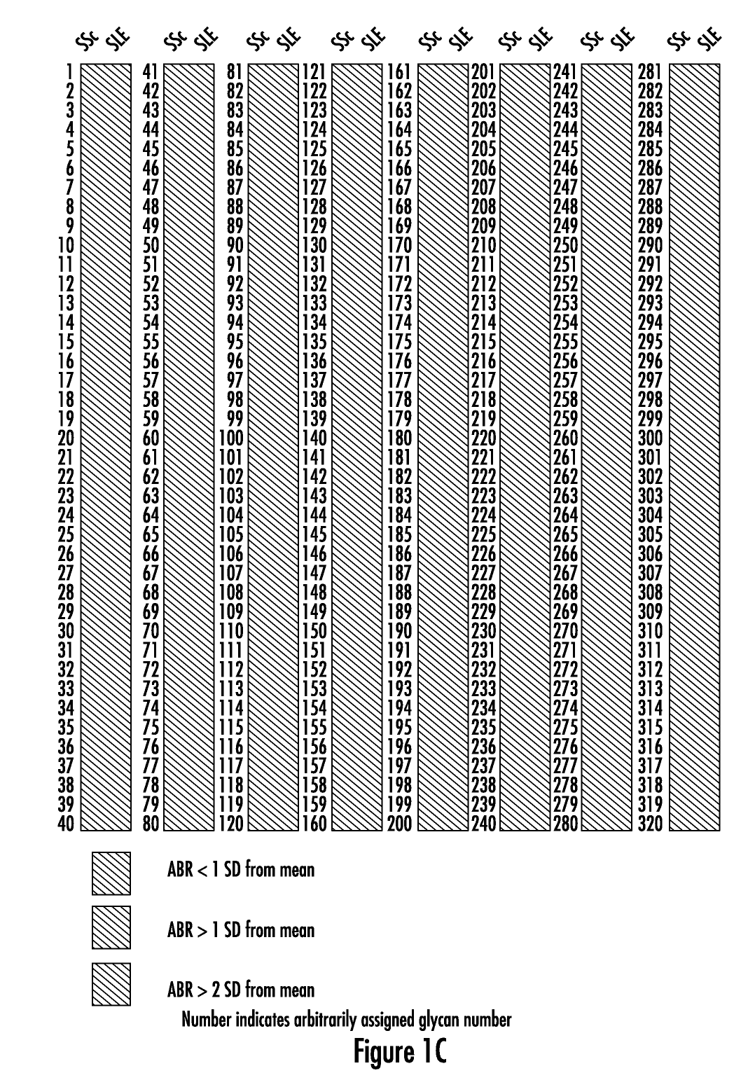

Glycosylation represents an important posttranslational modification that is involved in the regulation of many physiological processes. As a consequence, binding of antibodies to glycosylated structures in vivo may interfere with their function. In initial experiments we investigated whether sera from patients with systemic sclerosis target distinct carbohydrate structures. Pooled sera from 40 randomly selected patients with scleroderma and healthy controls were evaluated by glycan array for specific binding to 320 distinct glycans available through the Consortium for Functional Glycomics, at the National Institute of General Medical Sciences (NIGMS). As demonstrated shown in FIG. 1A, there was no significant difference in the antibody binding ratio (ABR) between scleroderma and healthy control serum for the majority of glycans (mean ABR 1.54, SD 1.58). The antibody binding ratio was calculated by taking the average of all individual ABRs, calculating the ratio of the fluorescence average of controls and disease gives a slightly different ratio, but numbers are: 1473.8 (control), 2101.8 (Scleroderma), 2273.3 (SLE). However, 10 distinct glycans were highly targeted by scleroderma sera with binding ratios >2 standard deviations (SD) above the mean ABR and an ABR as high as 15-fold (see FIG. 1B).

Among those, 2 glycans demonstrated a high degree of structural similarity, 4-sulfated N-Acetyl-Lactosamine (LacNAc) and 4-6-di-sulfated LacNAc. We confirmed the high ABR to both antigens using MG as a second control group (data not shown). Furthermore, the ABR of scleroderma serum was compared to pooled serum from 40 patients with SLE (FIG. 1C). Interestingly, this revealed that scleroderma patients recognize a distinct set of glycans compared to SLE patients (see FIG. 1C). For 4-sulfated LacNAc, the ABR calculated from pooled SLE serum was several fold lower than in scleroderma (3.7 versus 13.7 fold).

### Example 4

### Antibodies to 4-Sulfated LacNAc are Frequent and of High-Titer in Patients with Scleroderma

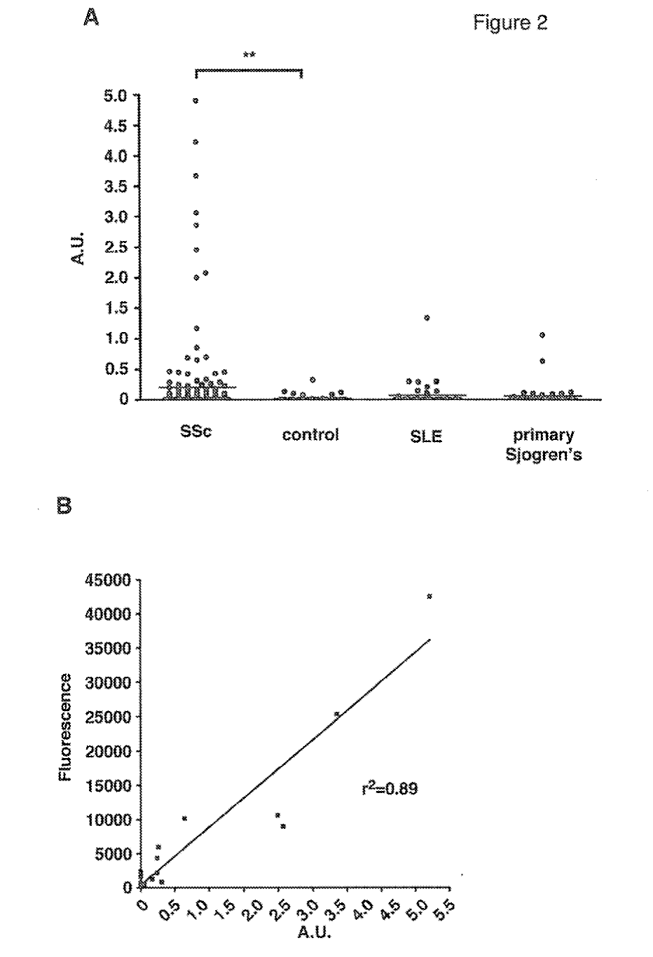

Subsequent studies focused on 4-sulfated LacNAc, since it was associated with a higher ABR compared to 4-6 di-sulfated LacNAc. However, similar studies can be performed to further characterize antibody binding to any of the glycans identified in the screen to be useful for the diagnosis of SLE, scleroderma, or both. An ELISA was developed using biotinylated 4-sulfated LacNAc as a capture antigen to determine the frequency and titer of antibodies in a large number of individual patients with scleroderma, as well as disease and healthy controls. As shown in FIG. 2, anti-4 sulfated LacNAc antibodies are frequent and of high-titer in scleroderma. Twenty seven of 181 (14.9%) of randomly selected scleroderma patients were positive for these antibodies, compared to 1/40 (2.5%) of healthy controls. The antibodies were less frequent in SLE (4/40; 10.0%). Importantly, anti-4-sulfated LacNAc antibody positive SLE disease controls exhibited relatively low titer antibodies compared to patients with scleroderma (mean A.U. 0.55; 0.84 and 1.25, respectively). Therefore, although the presence of the antibodies can be used for the diagnosis of either disease, the titer of the antibodies can further suggest the specific autoimmune disease of the patient.

These results were in agreement with our initial glycan array data using pooled sera from patients with scleroderma and SLE, which showed a relative specificity for scleroderma with an ABR of 13.7 compared to 3.7 (FIGS. 1B and C). Individual scleroderma patient sera (9 anti-4-sulfated LacNAc positive and 8 antibody negative) were selected for glycan array in order to obtain individual glycan profiles. There was a good correlation between the fluorescence data obtained by glycan array and the A.U. established by ELISA with an r² of 0.89 (see FIG. 2B).

### Example 5

### Sulfation at Position 4 of Galactose Plays a Dominant Role for Immunoreactivity Towards LacNAc

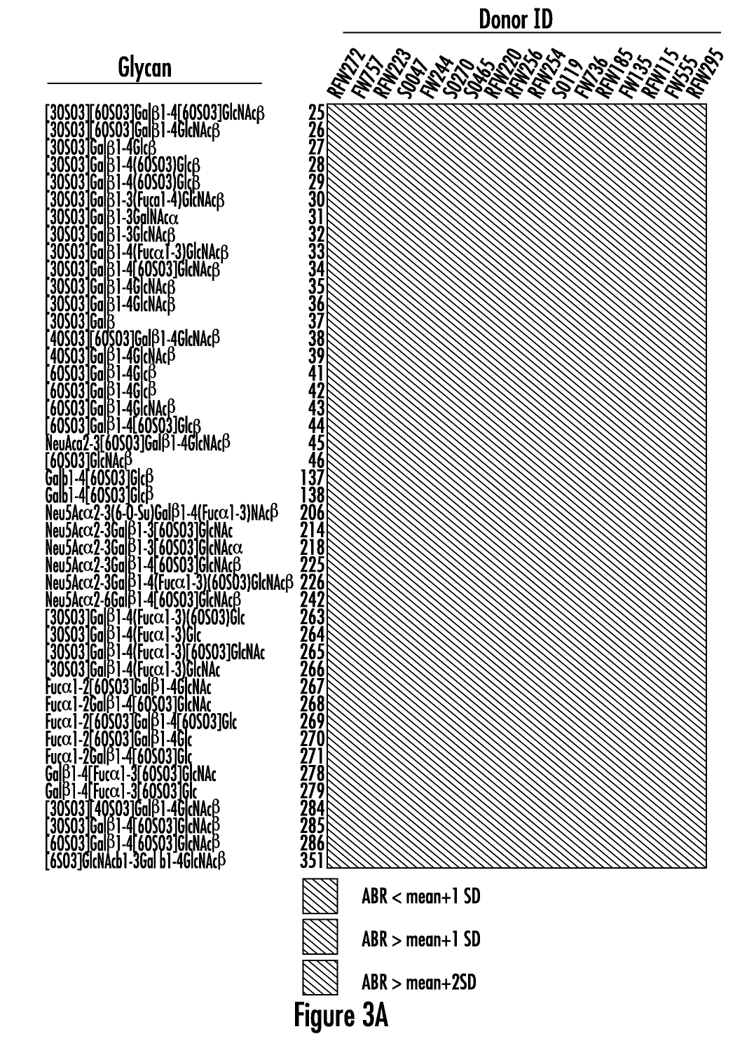

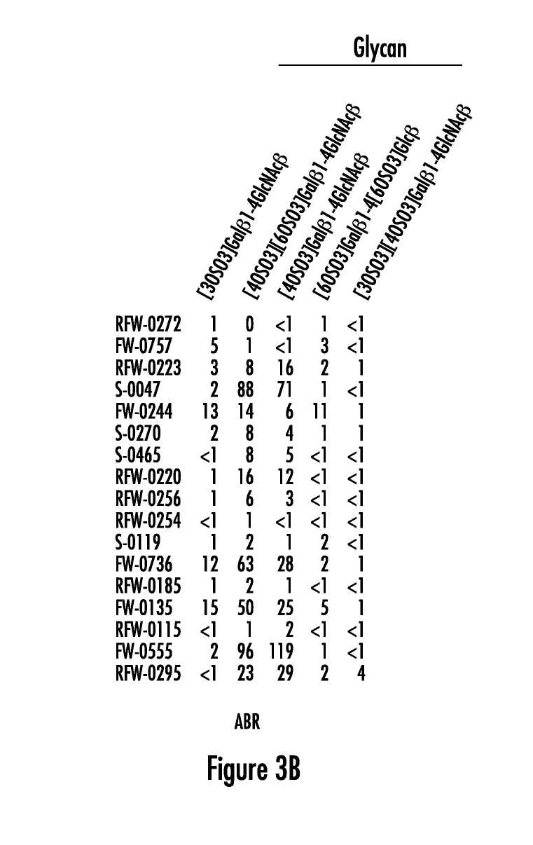

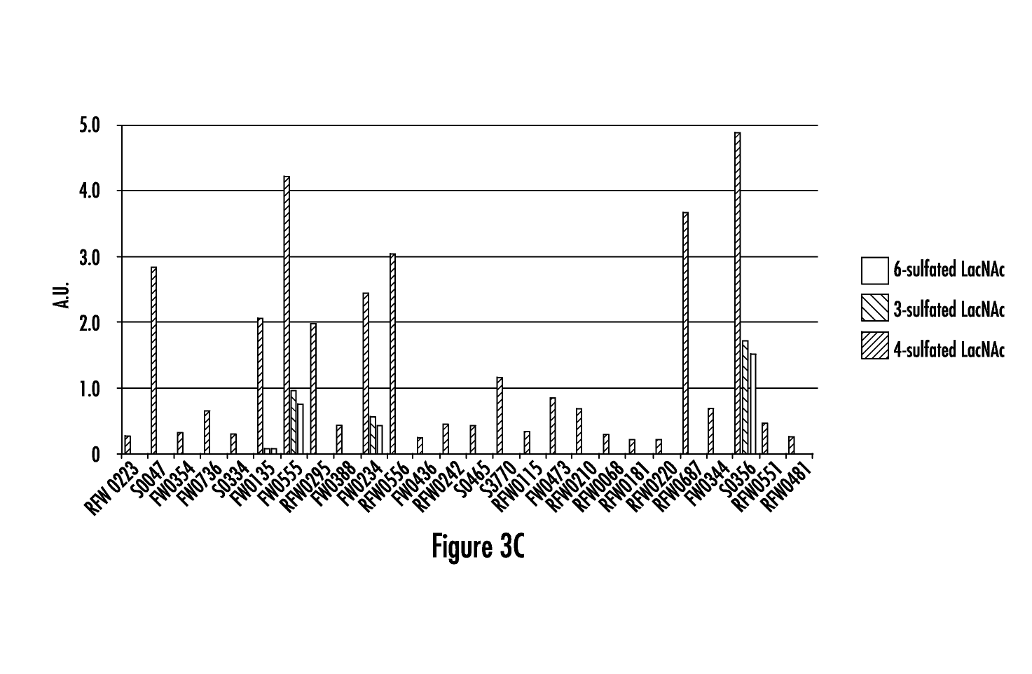

Modification of LacNAc by sulfation adds a negative charge to the glycan structure and may thereby lead to a non-specific increase in antibody binding. Therefore, differential binding of pooled scleroderma sera to various sulfate-containing glycans was analyzed. Glycan array CFG 3.0 contained 41 sulfated structures as shown in FIG. 3A. Interestingly, the mean ABR between sera from scleroderma and healthy controls was quite similar in this subgroup (mean 1.77, SD 2.24) compared to the overall ABR. 4-sulfated LacNAc as well as 4-6-di-sulfated LacNAc were the only sulfated glycans that showed an ABR higher than 2 SD above the mean. In particular, sulfation at position 3 or 6 of LacNAc led to a marked decrease in ABR (3.1 and 0.9, respectively) and LacNAc by itself was not recognized as an scleroderma antigen (ABR 1.8). Surprisingly, disulfation at position 3 and 4 of LacNAc completely abolished antibody binding (ABR 1.3), whereas disulfation at position 4 and 6 contributed to antigenicity (ABR 7.8). We confirmed these findings in a second glycan array using 17 preselected sera from scleroderma patients according to their anti-4 sulfated LacNAc status determined by ELISA (9 positive and 8 negative sera). As shown in FIG. 3B, ABRs were highest for 4-sulfated and 4-6-di-sulfated LacNAC (up to 119 fold) and extended in some patients to other sulfated glycans, although to a lesser degree. For 3- and 6-sulfated LacNAc these findings were also confirmed by ELISA (FIG. 3C). In summary, this data strongly suggests that differential sulfation of LacNAc plays an important role for immunogenicity of the glycan.

### Example 6

### Increased Prevalence of Pulmonary Vascular Disease, Sicca, and Raynaud's Disease in Anti-4 Sulfated LacNAc Positive Scleroderma Patients

The prevalence of antibodies to 4-sulfated LacNAc among 181 scleroderma patients was 14.9 percent using 3 SD above the mean of healthy controls as a cut-off. A detailed analysis of the clinical, serological and sociodemographic phenotype of these patients is shown in Table 2. There was no difference in disease type (diffuse versus limited), disease duration, age, gender, race, skin score, gastrointestinal and kidney involvement or mortality between anti-4 sulfated LacNAc positive and negative patients. Interestingly, scleroderma patients with serum antibodies against 4-sulfated LacNAc had a higher prevalence of pulmonary vascular disease (14/27 or 56% compared to 49/154 or 32%, p=0.02). Similarly, the lung severity score was higher in antibody positive patients (1.9 versus 1.4, p=0.02). There was no significant difference between the two groups for % predicted FVC and DLCO.

The prevalence of sicca symptoms was increased significantly in the antibody-positive group (74% versus 44%, p=0.006). Interestingly, the Raynaud's severity score was significantly lower in antibody positive patients (1.5 versus 2.0, p=0.016). There was no association between anti-4 sulfated LacNAc positivity and antibodies to topoisomerase I or centromere protein, respectively. In multivariate analysis corrected for age, gender, race and disease type, all associations remained significant. For patients with anti-4 sulfated LacNAc antibodies, the odds ratio for pulmonary vascular disease was 2.6 (CI 1.1-6.3), while for sicca it was 3.0 (CI 1.2-8.2). Manifestation of Raynaud's syndrome remained less severe with an odds ratio of 0.5 (CI 0.3-0.9).

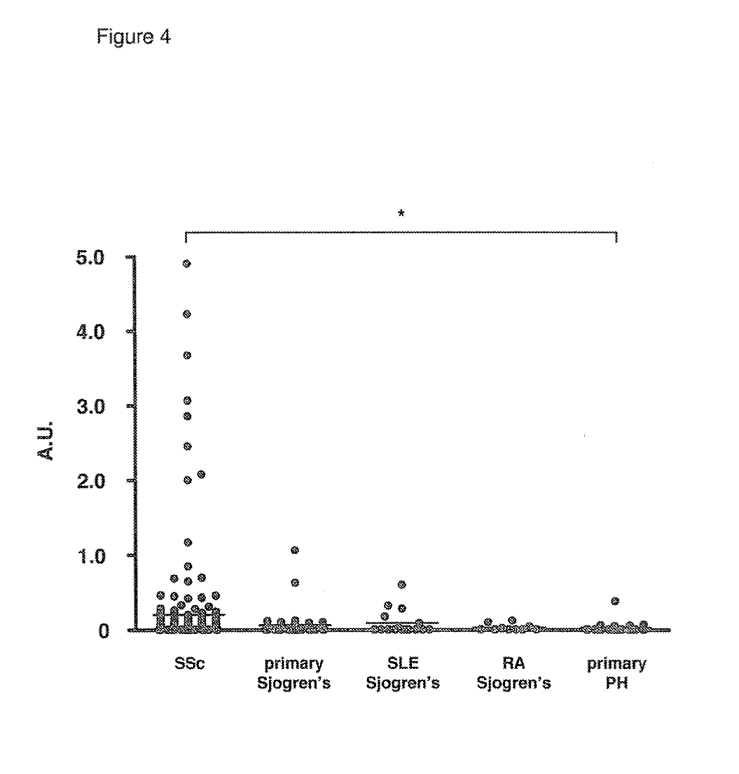

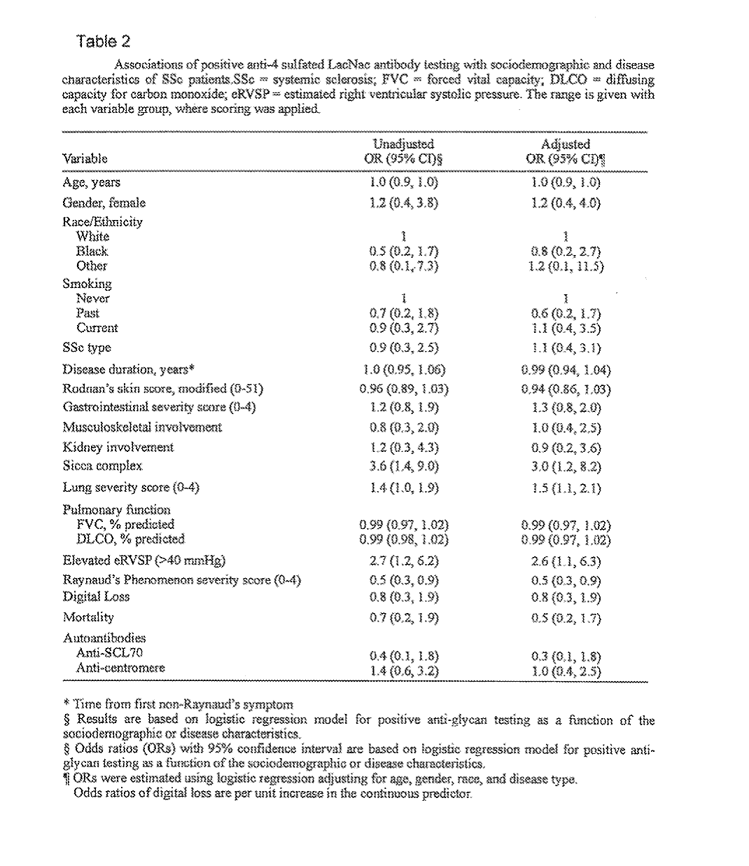

Studies were performed to determine whether anti-4 sulfated LacNAc antibodies are associated with sicca symptoms or pulmonary vascular disease in patient groups other than scleroderma; e.g. in 40 patients with primary Sjogren's, 20 SLE patients with secondary Sjogren's syndrome as well as 25 patients with primary pulmonary hypertension. Patients with either primary Sjogren's syndrome or secondary Sjogren's syndrome associated with SLE did not demonstrate an increased frequency in anti-4 sulfated LacNAc positivity compared to patients with systemic sclerosis (FIG. 4). There was no increased frequency in anti-4 sulfated LacNAc antibody positivity among patients with primary pulmonary hypertension (1/25; 4.0%).

All references, patents, patent applications, and GenBank numbers as of the date of filing of the instant application are hereby incorporated by reference as if they were each incorporated individually.
1. Wigley, F. M., Vascular disease in scleroderma. Clin Rev Allergy Immunol, 2009. 36(2-3): p. 150-75.
2. Harris, M. L. and A. Rosen, Autoimmunity in scleroderma: the origin, pathogenetic role, and clinical significance of autoantibodies. Cur Opin Rheumatol, 2003.15 (6): p. 778-84.
3. Mihai, C. and J. W. Tervaert, Anti-endothelial cell antibodies in systemic sclerosis. Ann Rheum Dis, 2010. 69(2): p. 319-24.
4. Chizzolini, C., et al., Autoantibodies to fibroblasts induce a proadhesive and proinβammatory fibroblast phenotype in patients with systemic sclerosis. Arthritis Rheum, 2002. 46(6): p. 1602-13.
5. Baroni, S. S., et al., Stimulatory autoantibodies to the PDGF receptor in systemic sclerosis. N Engl J Med, 2006. 354(25): p. 2667-76.
6. Loizos, N., et al., Lack of detection of agonist activity by antibodies to platelet-derived growth factor receptor alpha in a subset of normal and systemic sclerosis patient sera. Arthritis Rheum, 2009. 60(4): p. 1145-51.
7. Sperandio, M., C. A. Gleissner, and K. Ley, Glycosylation in immune cell trafficking. Immunol Rev, 2009. 230(1): p. 97-113.
8. Stringer, S. E., The role of heparan sulphate proteoglycans in angiogenesis. Biochem Soc Trans, 2006. 34(Pt 3): p. 451-3.
9. Yayon, A., et al., Cell surface, heparin-like molecules are required for binding of basic fibroblast growth factor to its high affinity receptor. Cell, 1991. 64(4): p. 841-8.
10. Rapraeger, A. C., A. Krufka, and B. B. Olwin, Requirement of heparan sulfate for bFGF-mediated fibroblast growth and myoblast differentiation. Science, 1991.252(5013): p. 1705-8.
11. Medsger, T. A., Jr., et al., A disease severity scale for systemic sclerosis: development and testing. J Rheumatol, 1999. 26(10): p. 2159-67.
12. Clements, P., et al., Inter and intraobserver variability of total skin thickness score (modified Rodnan TSS) in systemic sclerosis. J Rheumatol, 1995. 22(7): p. 1281-5.
13. Lung function testing: selection of reference values and interpretative strategies. American Thoracic Society. Am Rev Respir Dis, 1991. 144(5): p. 1202-18.
14. Denton, C. P., et al., Comparison of Doppler echocardiography and right heart catheterization to assess pulmonary hypertension in systemic sclerosis. Br J Rheumatol, 1997. 36(2): p. 239-43.
15. Blixt, O., et al., Printed covalent glycan array for ligand profiling of diverse glycan binding proteins. Proc Natl Acad Sci USA, 2004. 101(49): p. 17033-8.
16. Fillit, H. and R. Lahita, Antibodies to vascular heparan sulfate proteoglycan in patients with systemic lupus erythematosus. Autoimmunity, 1991. 9(2): p. 159-64.
17. Gyorgy, B., et al., Natural autoantibodies reactive with glycosaminoglycans in rheumatoid arthritis. Arthritis Res Ther, 2008. 10(5): p. R110.
18. Hylkema, M. N., et al., A new ELISA for the detection of anti-heparan sulfate reactivity, using photobiotinylated antigen. J Immunol Methods, 1994. 176(1): p. 33-43.
19. Lucas, A. H., et al., Carbohydrate Moieties as Vaccine Candidates: meeting summary. Vaccine, 2010. 28(4): p. 1121-31.
20. Sugahara, K., et al., Structural studies on sulfated glycopeptides from the carbohydrate-protein linkage region of chondroitin 4-sulfate proteoglycans of swarm rat chondrosarcoma. Demonstration of the structure Gal(4-O-sulfate)beta 1-3Gal beta 1-4XYL beta 1-O-Ser. J Biol Chem, 1988. 263(21): p. 10168-74.
21. Walker, U. A., et al., Clinical risk assessment of organ manifestations in systemic sclerosis: a report from the EULAR Scleroderma Trials And Research group database. Ann Rheum Dis, 2007. 66(6): p. 754-63.
22. Okano, Y., V. D. Steen, and T. A. Medsger, Jr., Autoantibody to U3 nucleolar ribonucleoprotein ( [symbol] brillarin) in patients with systemic sclerosis. Arthritis Rheum, 1992. 35(1): p. 95-100.
23. Negi, V. S., et al., Antiendothelial cell antibodies in scleroderma correlate with severe digital ischemia and pulmonary arterial hypertension. J Rheumatol, 1998. 25(3): p. 462-6.
24. Tamby, M. C., at al., Anti-endothelial cell antibodies in idiopathic and systemic sclerosis associated pulmonary arterial hypertension. Thorax, 2005.60(9): p. 765-72.
25. Hassoun, P. M., et al., Inflammation, growth factors, and pulmonary vascular remodeling. J Am Coll Cardiol, 2009. 54(1 Suppl): p. S10-9.
26. Steen, V. and T. A. Medsger, Jr., Predictors of isolated pulmonary hypertension in patients with systemic sclerosis and limited cutaneous involvement. Arthritis Rheum, 2003. 48(2): p. 516-22.
27. Stupi, A. M., et al., Pulmonary hypertension in the CREST syndrome variant of systemic sclerosis. Arthritis Rheum, 1986. 29(4): p. 515-24.
28. Chang, B., et al., Natural history of mild-moderate pulmonary hypertension and the risk factors for severe pulmonary hypertension in scleroderma. J Rheumatol, 2006. 33(2): p. 269-74.

We claim:

1. A use of a glycan in the manufacture of an immunoassay for diagnosing an autoimmune disease in a subject comprising:
a glycan for contacting with a subject sample; and
detecting binding of an antibody in the subject sample to the glycan, wherein binding of the antibody to the glycan is diagnostic for an autoimmune disease.

2. The use of claim 1, wherein the autoimmune disease comprises scleroderma or systemic lupus erythematosus (SLE).

3. The use of claim 1 or 2, wherein the autoimmune disease comprises scleroderma and the glycan is selected from the group consisting of: a 4-sulfated LacNAc, a 4-6-disulfated LacNAc, [4OSO3][6OSO3]Galβ1-4GlcNAc; [4OSO3]Galβ1-4GlcNAc; [3OSO3]Galβ1-4[6OSO3]GlcNAc; [6OSO3]Galβ1-4GlcNAc; Galβ1-4GlcNAcβ1-3Galβ1-4Glc; Galβ1-3GlcNAc 1-3Galβ1-4Glc; Galα1-4Galβ1-4Glc; Galβ1-3GalNAcβ1-3Galα1-4Galβ1-4Glc; Manα1-2Manα1-3(Manα1-2Manα1-6)Man; Manα1-2Manα1-2Manα1-3(Manα1-2Manα1-6(Manα1-3)Manα1-6)Man; Manα1-2Manα1-3Man; Neu5Acα2-3Galβ1-4Glc; Neu5Acα2-8Neu5Acα2-8Neu5Acα2-3(GalNAcβ1-4)Galβ1-4GlcNeu5Acα2-3Galβ1-4GlcNAc 1-3Galβ1-4GlcNAc; Neu5Acα2-3(GalNAcβ1-4)Galβ1-4GlcNAc; Neu5Acα2-8Neu5Acα2-3(GalNAcβ1-4)Galβ1-4Glc; Neu5Acα2-6Galβ1-4GlcNAc; Neu5Acα2-8Neu5Ac; and Neu5Acα2-3(GalNAcβ1-4)Galβ1-4Glc; or any combination thereof.

4. The use of any of claims 1-3, wherein the autoimmune disease comprises scleroderma and the glycan is selected from the group consisting of 4-sulfated LacNAc, a 4-6-disulfated LacNAc, [4OSO3][6OSO3]Galβ1-4GlcNAc; and [4OSO3]Galβ1-4GlcNAc; or any combination thereof.

5. The use of claim 4, wherein the wherein binding of the antibody to the glycan is diagnostic for one or more conditions selected from the group consisting of pulmonary hypertension and sicca; or a combination thereof.

6. The use of claim 4, wherein the binding of the antibody to the glycan is diagnostic for a less severe Raynaud's phenotype.

7. The use of any of claims 1 to 3, wherein the autoimmune disease comprises SLE and the glycan is selected from the group consisting of [4OSO3][6OSO3]Galβ1-4GlcNAc; [4OSO3]Galβ1-4GlcNAc; [3OSO3]Galβ1-4[6OSO3]GlcNAc; [6OSO3]Galβ1-4GlcNAc; Manα1-2Manα1-3Man; Neu5Acα2-3(GalNAcβ1-4)Galβ1-4GlcNAc; Neu5Acα2-8Neu5Acα2-3(GalNAcβ1-4)Galβ1-4Glc; Neu5Acα2-6Galβ1-4GlcNAc; Neu5Acα2-8Neu5Ac; and Neu5Acα2-3(GaINAcβ1-4)Galβ1-4Glc.

8. The use of any one of claims 1 to 3, wherein the autoimmune disease comprises SLE or scleroderma and the glycan is selected from the group consisting of [4OSO3][6OSO3]Galβ1-4GlcNAc; [4OSO3]Galβ3-4GlcNAc; Manα1-2Manα1-3Man; Neu5Acα2-6Galβ1-4GlcNAc; Neu5Acα2-8Neu5Ac; and Neu5Acα2-3(GalNAcβ1-4)Galβ1-4Glc.

9. The use of any of claims 1 to 8, wherein the sample is selected from the group consisting of blood, scrum, and plasma.

10. The use of any of claims 1 to 9, wherein the binding is compared to a binding of a control sample.

11. The use of claim 10, wherein the control sample is selected from the group consisting of normal blood, normal serum, and intravenous immunoglobulin (IVIG); or a combination thereof.

12. A method for diagnosing an autoimmune disease in a subject comprising:
contacting a subject sample with a glycan; and
detecting binding of an antibody in the subject sample to the glycan, wherein binding of the antibody to the glycan is diagnostic for an autoimmune disease.

13. The method of claim 12, wherein the autoimmune disease comprises scleroderma or systemic lupus erythematosus (SLE).

14. The method of claim 12 or 13, wherein the autoimmune disease comprises scleroderma and the glycan is selected from the group consisting of: a 4-sulfated LacNAc, a 4-6-disulfated LacNAc, [4OSO3][6OSO3]Galβ1-4GlcNAc; [4OSO3]Galβ1-4GlcNAc; [3OSO3]Galβ1-4[6OSO3]GlcNAc; [6OSO3]Galβ1-4GlcNAc; Galβ1-4GlcNAcβ1-3Galβ1-4Glc; Galβ1-3GlcNAcβ1-3Galβ1-4Glc; Galα1-4Galβ1-4Glc; Galβ1-3GalNAcβ1-3Galα1-4Galβ1-4Glc; Manα1-2Manα1-3(Manα1-2Manα1-6)Man; Manα1-2Manα1-2Manα1-3(Manα1-2Manα1-6(Manα1-3)Manα1-6)Man; Manα1-2Manα1-3Man; Neu5Acα2-3Galβ1-4Glc; Neu5Acα2-8Neu5Acα2-8Neu5Acα2-3(GalNAcβ1-4)Galβ1-4GlcNeu5Acα2-3Galβ1-4GlcNAcβ1-3Galβ1-4GlcNAc; Neu5Acα2-3(GalNAcβ1-4)Galβ1-4GlcNAc; Neu5Acα2-8Neu5Acα2-3(GalNAcβ1-4)Galβ1-4Glc; Neu5Acα2-6Galβ1-4GlcNAc; Neu5Acα2-8Neu5Ac; and Neu5Acα2-3(GalNAcβ1-4)Galβ1-4Glc; or any combination thereof.

15. The method of any of claims 12 to 14, wherein the autoimmune disease comprises scleroderma and the glycan is selected from the group consisting of 4-sulfated LacNAc, a 4-6-disulfated LacNAc, [4OSO3][6OSO3]Galβ1-4GlcNAc; and [4OSO3]Galβ1-4GlcNAc; or any combination thereof.

16. The method of claim 15, wherein the wherein binding of the antibody to the glycan is diagnostic for one or more conditions selected from the group consisting of: pulmonary hypertension and sicca; or a combination thereof.

17. The method of claim 15, wherein the binding of the antibody to the glycan is diagnostic for a less severe Raynaud's phenotype.

18. The method of any of claims 12 to 14, wherein the autoimmune disease comprises SLE and the glycan is selected from the group consisting of [4OSO3][6OSO3]Galβ1-4GlcNAc; [4OSO3]Galβ1-4GlcNAc; [3OSO3]Galβ1-4[6OSO3]GlcNAc; [6OSO3]Galβ1-4GlcNAc; Manα1-2Manα1-3Man; Neu5Acα2-3(GalNAcβ1-4)Galβ1-4GlcNAc; Neu5Acα2-8Neu5Acα2-3(GalNAcβ1-4)Galβ1-4Glc; Neu5Acα2-6Galβ1-4GlcNAc; Neu5Acα2-8Neu5Ac; and Neu5Acα2-3(GalNAcβ1-4)Galβ1-4Glc.

19. The method of any one of claims 12 to 14, wherein the autoimmune disease comprises SLE or scleroderma and the glycan is selected from the group consisting of [4OSO3][6OSO3]Galβ1-4GlcNAc; [4OSO3]Galβ1-4GlcNAc; Manα1-2Manα1-3Man; Neu5Acα2-6Galβ1-4GlcNAc; Neu5Acα2-8Neu5Ac; and Neu5Acα2-3(GalNAcβ1-4)Galβ1-4Glc.

20. The method of any of claims 12 to 19, wherein the sample is selected from the group consisting of blood, serum, and plasma.

21. The method of any of claims 12 to 20, wherein the binding is compared to a binding of a control sample.

22. The method of claim 21, wherein the control sample is selected from the group consisting of normal blood, normal serum, normal plasma, and WIG; or a combination thereof.

23. The method of claim 12, further comprising obtaining a subject sample.

24. A kit for the use of any of claims 1 to 11 or practicing the method of any of claims 12 to 23.
